(19) United States
(12) Patent Application Publication
(10) Pub. No.: US 2022/0047785 A1
(43) Pub. Date: Feb. 17, 2022

(54) BREAST PUMP

(71) Applicant: MEDELA HOLDING AG, Baar (CH)

(72) Inventor: Andrea Masucci, Männedorf (CH)

(21) Appl. No.: 17/279,076

(22) PCT Filed: Sep. 23, 2019
(86) PCT No.: PCT/EP2019/075464

§ 371 Date: Mar. 23, 2021

(30) Foreign Application Priority Data
Sep. 27, 2018 (EP) 18197066.6

(51) Int. Cl. A61M 1/06; A61M 1/00; G16H 40/63

(52) U.S. Cl. CPC A61M 1/06; A61M 1/064; A61M 1/73; G16H 40/63; A61M 2205/6036; A61M 2205/581; A61M 2205/80; A61M 2205/583; A61M 2210/1007; A61M 2205/502

(57) ABSTRACT

An electrically operated breast pump for pumping human breast milk comprises an electrical pump unit (15) and an electronic control unit (14) for controlling the pump unit (15). The pump unit (15) can be activated by the control unit (14) in a time-delayed manner. The breast pump according to the invention allows a stress-free start of the process of pumping breast milk in a simple manner.

### TECHNICAL FIELD

The present invention relates to an electrically operated breastpump for expressing human breastmilk.

### PRIOR ART

Breastpumps for expressing human breastmilk are well known. Essentially, there are two different types: the first is manually operated, i.e. the vacuum required for expressing is generated by manual actuation of a vacuum pump. In the second type, an electric pump assembly is present, having an electric motor for generating the necessary vacuum.

Electrically operated breastpumps are designed with varying degrees of complexity in terms of their control. There are single-action breastpumps, which apply a constant pulsating vacuum to the breastpump, wherein the pump frequency and the suction strength can normally be selected. Other breastpumps can be programmed with relatively complex suction curves in order to generate a suction behavior which is as lifelike as possible. Many of these pumps moreover offer the option of expressing from both breasts at the same time. To this end, both breast shields firstly have to be placed tightly on the breast and only then does the breastpump begin to operate in an optimum manner.

Immediately before expressing, the mother is therefore busy positioning the breast shields optimally on both breasts. Consequently, she does not have one hand free to activate the breastpump. However, if she firstly switches on the breastpump and only then places the breast shields on the breast, the vacuum already generated prevents the breast shields from being placed on both breasts in an optimum and pain-free manner for the mother.

A breastpump unit is known from WO 2008/009145, in which a signal transmitting unit is arranged in the breast shield and a receiver unit is arranged in the pump housing. The receiver unit is connected to the control of the pump assembly. The mother can thus alter the setting of the breastpump, i.e. the pump frequency and the suction strength, i.e. the vacuum applied, whilst expressing. Nevertheless, the mother must be ready and the breast shields must be correctly applied when the breastpump is switched on.

### PRESENTATION OF THE INVENTION

It is therefore an object of the invention to provide a breastpump which enables the mother to begin expressing in an optimum and stress-free manner.

This object is achieved by a breastpump having the features of claim 1.

The inventive electrically operated breastpump for expressing human breastmilk has an electric pump assembly and an electronic control unit for controlling the pump assembly. According to the invention, the pump assembly can be activated in a time-delayed manner by means of the control unit.

Thanks to this time delay, the mother can firstly switch on the breastpump and then calmly place the breast shields on her breast and find the optimum position there. The mother therefore gains time between the moment at which the breastpump is switched on and the moment at which the expressing procedure begins.

This time delay or this gain in time is not necessarily always desired. Experienced mothers or mothers who only express from one breast, and therefore have one hand free for switching on the breastpump, might prefer the breastpump to switch on directly. Therefore, in preferred embodiments, it is possible to select whether the pump assembly is activated immediately when the breastpump is switched on or in a time-delayed manner. Therefore, the time delay can preferably be activated or deactivated by a user. This means either the breast shield starts immediately as standard and the suction procedure only begins later if there has been an individual activation of the time delay, or the breast shield starts in a time-delayed manner as standard and the mother has the option of switching off this delay.

The breastpump preferably has a switch which can be actuated by a user for the purpose of activating the time delay. The term “switch” should be understood in a broad sense here; it includes tumbler switches, push buttons, rotary switches, pressure-sensitive areas and all other forms of actuating means which are manually actuated in order to relay a start or stop signal to a control. This also includes a start signal by means of a speech recognition module or a different type of signal generator.

The breastpump preferably has a start switch for activating the pump assembly, wherein the switch for activating the time delay is the same switch. In other embodiments, the switch for activating the time delay is a different switch.

The switch for activating the time delay is preferably arranged on or in the pump housing in which the pump assembly and the control unit are arranged. This is a simple and cost-effective variant.

Alternatively or additionally, the breastpump has a speech recognition module for activating the time delay via a user. The mother is therefore still able to decide whilst applying the breast shields whether or not she would like a time delay.

Alternatively or additionally, the breast shield has a switch connected to the control unit for the purpose of activating the time delay. This also enables the mother to activate the time delay whilst applying the breast shield.

The said switch and the speech recognition module can be alternatively or additionally used to deactivate or prematurely stop the time delay.

So that the mother knows how much time she has left before the start of the suction procedure, a display unit is preferably present, which, during the time delay, issues a signal which can be perceived by a user. The signal is preferably optical and/or acoustic.

An optical display unit is preferably present, which, during the time delay, gives an indication as to when the pump assembly will start. For example, it is a changing clock or a changing bar. The clock is represented in analog or digital form, for example. It preferably counts backwards. A bar preferably becomes steadily smaller or larger and/or the colors change. The display unit can also be a decreasing numerical increment. In other embodiments, an acoustic signal can be heard at increasingly shorter intervals and/or it increases or decreases in volume.

So that a mother does not have to wait when she is ready to express, a means is preferably present for prematurely terminating an activated time delay. The means is preferably a switch, for example a speech recognition module. It can be the same switch as that used to activate the time delay. It can also be a different switch.

In simple embodiments, the duration of the time delay is predetermined and cannot be changed. In other embodiments, it is possible for the mother to select the duration at the start, for example by pressing the switch multiple times, by rotating it if the switch is a rotary button or by setting the duration via another input means, for example via a separate switch or rotary button.

Further embodiments are revealed in the dependent claims.

### BRIEF DESCRIPTION OF THE DRAWINGS

A preferred embodiment of the invention is described below with reference to the drawings, which serve merely for explanation and should not be interpreted as restrictive. In the drawings:

FIG. 1 shows a schematic illustration of an inventive breastpump with a flow chart which shows the control;

FIG. 2 shows a schematic illustration of a housing containing the breastpump according to FIG. 1.

### DESCRIPTION OF PREFERRED EMBODIMENTS

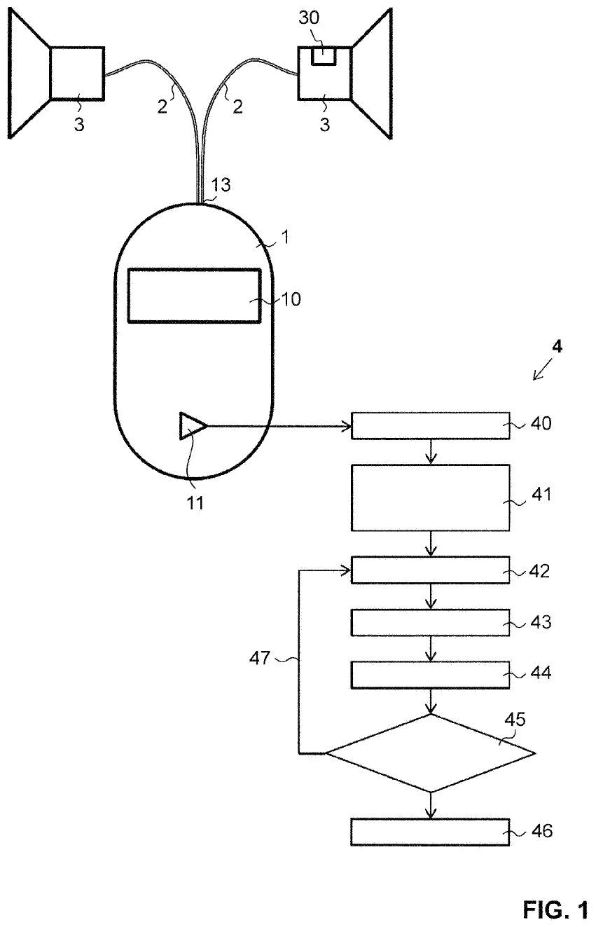

FIG. 1 shows an inventive breastpump having a housing 1 and a suction connector 13. Two suction tubes 2 are inserted in the suction connector 13, each leading to a breast shield 3.

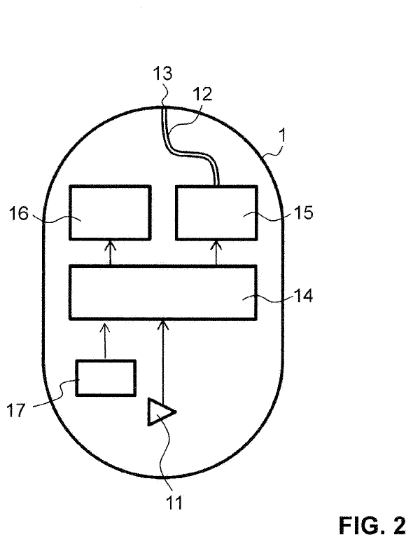

A pump assembly 15 having an electric motor, a control unit 14 for controlling the pump assembly 15 and a display control 16 are arranged in the housing 1, as can be seen in FIG. 2. A connecting channel 12, normally a tube, leads from the pump assembly 15 to the suction connector 13. In other embodiments, the pump assembly itself ends at the suction connector 13. The control unit 14 has a time delay module, for example a circuit with one or more relays.

A display unit, here in the form of a display 10, is furthermore arranged on or in the housing 1, as can be seen in FIG. 1. A switch 11 is furthermore present. In this example, the switch 11 serves for switching the breastpump on and off and, at the same time, for activating the time delay.

The mode of operation can be seen with the aid of the flow chart. If the switch 11 is actuated by the mother, the pump starts immediately. However, if the switch 11 is pressed for somewhat longer, a brief acoustic signal sounds. The mother is thus aware that the time delay has been activated. The control 14 therefore switches to time delay mode. This is the start 40.

The control 14, which is connected to the display control 16 or incorporates this, now activates the display 10. A number, for example the number 10, appears on the display 10. This is the first step 41.

In a second step 42, the control 14 waits a defined time, for example 1 second, and then reduces its decrement and subsequently shows a decreased number on the display, the number 9 here. The optical display therefore shows a decreasing numerical increment. These are the third and fourth steps 43 and 44. It is checked in the decision 45 whether the predefined duration of the delay has been fulfilled. If yes, the end of the delay 46 has been reached and the control unit starts the pump assembly. If the duration has not yet been reached, the second to fourth steps 42, 43, 44 are repeated in a loop 47. Depending on the embodiment, a possible premature deactivation of the time delay can likewise be implemented by means of this switch 11.

Alternatively or additionally, in some embodiments, at least one of the breast shields is designed with a signal generator 30, here a switch, in order to either prematurely deactivate the time delay and/or switch on or switch off the time delay before the expressing procedure, depending on the embodiment.

In other embodiments, a speech recognition module 17 is alternatively or additionally arranged in or on the housing 1, which is likewise connected to the control unit 14.

In a simple manner, the inventive breastpump enables a stress-free start when expressing breastmilk.

LIST OF REFERENCE SIGNS

| | |
|---|---|
| 1 | Breastpump |
| 10 | Display |
| 11 | Switch |
| 12 | Connecting channel |
| 13 | Suction connector |
| 14 | Control unit |
| 15 | Pump assembly |
| 16 | Display control |
| 17 | Speech recognition module |
| 2 | Suction tube |
| 3 | Breast shield |
| 30 | Signal generator |
| 4 | Flow chart of the control for the time delay |
| 40 | Start delay |
| 41 | First step |
| 42 | Second step |
| 43 | Third step |
| 44 | Fourth step |
| 45 | Decision |
| 46 | End of the delay |
| 47 | Loop |

1. An electrically operated breastpump for expressing human breastmilk having an electric pump assembly and an electronic control unit for controlling the pump assembly, wherein the pump assembly can be activated in a time-delayed manner by means of the control unit.

2. The breastpump as claimed in claim 1, wherein the time delay can be activated or deactivated by a user.

3. The breastpump as claimed in claim 1, wherein it has a switch which can be actuated by a user for the purpose of activating the time delay.

4. The breastpump as claimed in claim 3, wherein it has a start switch for activating the pump assembly and wherein the switch for activating the time delay is the switch for activating the pump assembly.

5. The breastpump as claimed in claim 3, further comprising a start switch for activating the pump assembly and wherein the switch for activating the time delay is a different switch.

6. The breastpump as claimed in claim 3, wherein the pump assembly and the control unit are arranged in a pump housing and wherein the switch for activating the time delay is arranged on or in the pump housing.

7. The breastpump as claimed in claim 1, wherein the breastpump has a speech recognition module for activating the time delay via a user.

8. The breastpump as claimed in claim 1, wherein the breastpump has at least one breast shield for applying to a human breast and wherein the breast shield has a switch connected to the control unit for the purpose of activating the time delay.

9. The breastpump as claimed in claim 1, wherein a display unit is present, which, during the time delay, issues a signal which can be perceived by a user.

10. The breastpump as claimed in claim 1, wherein an optical display unit is present, which, during the time delay, gives an indication as to when the pump assembly will start.

11. The breastpump as claimed in claim 10, wherein the optical display unit shows a changing clock or a changing bar or a decreasing numerical increment.

12. The breastpump as claimed in claim 9, wherein the display unit issues an acoustic signal.

13. The breastpump as claimed in claim 1, wherein a means is present for prematurely terminating an activated time delay.

14. The breastpump as claimed in claim 13, wherein the means is a switch.

15. The breastpump as claimed in claim 1, wherein the duration of the time delay can be selected by the user.
